(19) United States
(12) Patent Application Publication
(10) Pub. No.: US 2011/0060390 A1
(43) Pub. Date: Mar. 10, 2011

(54) METHOD FOR BENDING THE SELF-REGULATING CABLE AND HEATING MAT FOR PROTECTING OVER-HEATING

(75) Inventors: Seo Gon Kim, Gyeonggi-do (KR); Kun Ho Yang, Gyeonggi-do (KR)

(73) Assignee: Solco Biomedical Co., Ltd., Gyeonggi-do (KR)

(21) Appl. No.: 12/297,192

(22) PCT Filed: Apr. 17, 2007
(86) PCT No.: PCT/KR2007/001863

§ 371 Date: Sep. 17, 2009

(30) Foreign Application Priority Data
Apr. 17, 2006 (KR) 10-2006-0034558
Jun. 7, 2006 (KR) 10-2006-0051017

(51) Int. Cl. A61F 7/08; H01R 43/00

(52) U.S. Cl. 607/98; 29/592.1

(57) ABSTRACT

Disclosed are a method for bending a self-regulating cable having a function of maintaining a predetermined temperature by applied electric power, in which the self-regulating cable is arranged in zigzags by easily bending the self-regulating cable, and a heating mat for preventing overheating using the same. A heating mat comprises: a heat generating means for maintaining a predetermined temperature with applied electric power; a control unit electrically connected to the heat generating means, for regulating the electric power supplied to the heat generating means; and an exterior material surrounding the heat generating means. The method comprises: setting a bent portion of the self-regulating cable; exposing the semi-conductive polymer by removing a portion of the insulation film, which corresponds to the bent portion; exposing a pair of wires by removing the exposed semi-conductor polymer; and bending portion of the pair of wires, which correspond to the exposed bent portion.

### TECHNICAL FIELD

The present invention relates to a method for bending a self-regulating cable and a heating mat for preventing overheating, and more particularly to a method for bending a self-regulating cable having a function of maintaining a predetermined temperature by applied electric power, in which the self-regulating cable is arranged in zigzags by easily bending the self-regulating cable, and a heating mat for preventing overheating using the same.

### BACKGROUND ART

In general, a human body often experiences harmful states such as muscular or nervous tension due to excessive exercise or stress. Especially, in this day and age, fatigue materials are left in the body due to nervous tension and stress, obstructing the flow of blood.

Especially, it is well known that a thermo-therapy using sand and a hot bath is effective for a curing effect and alleviation of pain. The thermo-therapy uses conduction heat or radiation heat to improve circulation of peripheral blood and to expedite metabolism, for various diseases caused by degeneration of the functions of a human body, articular rheumatism, neuralgia, chronic inflammation, recovery of fatigue. Accordingly, various heating devices have been developed and used together with various fomentation packs having a thermo-therapy effect.

The heating devices include one used by filling a liquid of a high temperature in a bag having a simple structure and easy to use and one heated by electricity by providing a heat generating means, i.e. a heater. However, since the bag cannot be used for a long time since the liquid heated to a high temperature is easily cooled down, a heating mat having a heating effect by using electricity has been suggested.

FIG. 1 is a partially cut-away perspective view illustrating a conventional heating mat using a heat generating means.

As illustrated in FIG. 1, in the conventional mat, an electric heating wire 30, which is a heat generating means, is arranged between interior materials 10 and 15 formed of fiber or a non-woven fabric and is surrounded in the interior of an exterior material 20. If a current is supplied though a control unit 40 electrically connected to the electric heating wire 30, heat is emitted from the electric heating wire 30.

The conventional heating mat can maintain the heated state for a long time and brings a curing effect using the electric heating wire 30. However, since the temperature felt by a user is continuously changed by the environmental temperature of the heating mat and the current applied to the electric heating wire 30, the curing effect decreases. Further, the control section 40 should be continuously regulated during use as the temperature increases or decreases.

Further, since the heat generated by the conventional mat is transferred while making contact with the skin of a user, the curing effect is restricted as the heat cannot penetrate the skin deeply.

The conventional heating mat generates heat by applying current to a resistance wire having a predetermined resistance value, but the electric heating wire 30 is deformed and generates partial overheating by continuous use, causing a serious safety problem. Especially, since the partial overheating of the electric heating wire 30 has the danger of causing a bodily accident such as a fire, a burn, or an electric shock, a basic measure is urgent.

### DISCLOSURE OF INVENTION

### Technical Problem

Therefore, the present invention has been made in view of the above-mentioned problems, and the present invention provides a heating mat for preventing overheating, which can prevent the heating mat from being overheated above a predetermined temperature regardless of the used environment.

Also, the present invention provides a heating mat for preventing overheating having a function of emitting a large amount of far infrared rays having an effect of curing a human body by the heating effect and of reducing electromagnetic waves.

Also, the present invention provides a heating mat for preventing overheating, capable of being conveniently used for a long time due to an excellent feeling of touch and absorption of a portion making contact with a human body.

Also, the present invention provides a method for bending a self-regulating cable capable of easily bending the self-regulating cable having a function of maintaining the periphery at a predetermined temperature.

### Technical Solution

In accordance with an aspect of the present invention, there is provided a heating mat for preventing overheating, comprising: a heat generating means for maintaining a predetermined temperature with applied electric power; a control unit electrically connected to the heat generating means, for regulating the electric power supplied to the heat generating means; and an exterior material surrounding the heat generating means.

The heat generating means is a self-regulating cable.

In the self-regulating cable, a pair of wires are arranged in the interior of an insulation film by the medium of a semi-conductive polymer.

The semi-conductive polymer comprises carbon.

In the self-regulating cable, a ground wire is formed on the exterior of the insulation film and the ground wire and the insulation film are surrounded by an external insulation film.

The heat generating means has a partially different thickness.

A portion of the heat generating means which has a small thickness is bent.

The heat generating means is exposed by removing a portion of the semi-conductive polymer and separating the pair of wires.

At least one of the pair of exposed wires is surrounded by another insulation film.

The pair of exposed wires are bent.

One of the pair of exposed wires is connected to the ground.

In the heat generating means, portions of the insulation film and the external insulation film, which are removed, are bent.

In the heat generating means, portions of the insulation film and the external insulation film, which are removed, are bent.

The heat generating means is exposed by removing a portion of the semi-conductive polymer and separating the pair of wires.

At least one of the pair of exposed wires is surrounded by another insulation film.

The ground wire separated from the pair of exposed wires is left.

The control unit comprises a current leakage interrupter detecting leaked current between the ground wire and the pair of wires.

In the heat generating means, a plurality of Self-regulating cables are connected in series by an engaging means.

The engaging means is connected in series to the pair of wires of the self-regulating cable to connect a plurality of self-regulating cables.

The engaging means is bent in the heat generating means.

The heating mat further comprises: an interior material surrounding the heat generating means in the interior of the exterior material.

Stone powder is provided between the exterior material and the interior material.

The stone powder comprises at least one material selected from a group consisting of germanium, tourmaline, loess, hardwood charcoal, crystal, elvan, and ceramic.

A surface of the exterior material is a jacquard material.

The heat generating means is connected in parallel to a wire connected through the control unit and is arranged in the interior of the exterior material.

In according to another aspect of the present invention, there is provided a method for bending a self-regulating cable comprising a pair of wires arranged in the interior of an insulation film by the medium of a semi-conductive polymer filled in the interior of the insulation film, the method comprising: setting a bent portion of the self-regulating cable; exposing the semi-conductive polymer by removing a portion of the insulation film, which corresponds to the bent portion; exposing a pair of wires by removing the exposed semi-conductor polymer; and bending portion of the pair of wires, which correspond to the exposed bent portion.

The self-regulating cable comprises a ground wire formed outside the insulation film and an external insulation film surrounding the ground wire and the insulation film, the method further comprising: removing a portion of the external film, which corresponds to the bent portion, after setting the bent portion of the self-regulating cable.

At least one of the pair of wires is surrounded by another insulation film after exposing the pair of wires.

### ADVANTAGEOUS EFFECTS

As mentioned above, the present invention has the following effects.

Since the heating mat for preventing overheating according to the present invention prevents overheating using a self-regulating cable having the characteristics in which generation of heat is stopped when the resistance of a heat generating means increases when the peripheral temperature increases, it can easily maintain a constant temperature regardless of the use condition and the peripheral environment and can be conveniently used.

Further, in the heating mat for preventing overheating according to the present invention, since a plurality of heat generating means are connected in a parallel structure, the current flowing through the heat generating means is reduced and generation of electromagnetic waves is restrained. Further, a large amount of far infrared rays having a curing effect for a human body by emitting far infrared rays are emitted by the carbon component of the semi-conductive polymer included in the heat generating means, thereby increasing the curing effect for a user.

Further, in the heating mat for preventing overheating according to the present invention, stone powder is used as an absorbing material, improving the feeling of touch at a portion making contact with a human body. Therefore, the heating mat can be used for a long time.

Further, the method of bending a self-regulating cable according to the present invention can easily bend the self-regulating cable having a function of maintaining the periphery at a constant temperature by deforming the cross-section of a bent portion by a small degree.

Further, in the heating mat for preventing overheating according to the present invention, if a power source is supplied to a semi-conductive polymer, since the temperature increases instantaneously, it can be conveniently used by increasing the temperature immediately even in winter season without inconvenience of waiting for more than a predetermined time.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a partially cut-away perspective view illustrating a conventional heating mat using a heat generating means;

FIG. 2 is a partially cut-away perspective view illustrating a heating mat for preventing overheating according to the first embodiment of the present invention;

FIG. 3 is a perspective view illustrating the interior structure of a self-regulating cable used as a heat generating means in the heating mat of the present invention;

FIG. 4 is a cross-sectional view of the self-regulating cable illustrated in FIG. 3;

FIG. 5 is a perspective view illustrating the bent shape of the heat generating means used in the heating mat for preventing overheating illustrated in FIG. 2;

FIG. 6 is a front view enlarging the bent portion of the heat generating means of FIG. 5;

FIG. 7 is a flow chart for explaining a method for bending a self-regulating cable according to an embodiment of the present invention;

FIG. 8 is a circuit connecting view illustrating a structure in which a heat generating means is connected in parallel in a heating mat according to another embodiment of the present invention;

FIG. 9 is a plan view displaying positions where electromagnetic waves are measured in the heating mat illustrated in FIGS. 8; and

FIG. 10 is a table displaying data showing a result obtained by measuring electromagnetic waves in mG (m Gauss) unit.

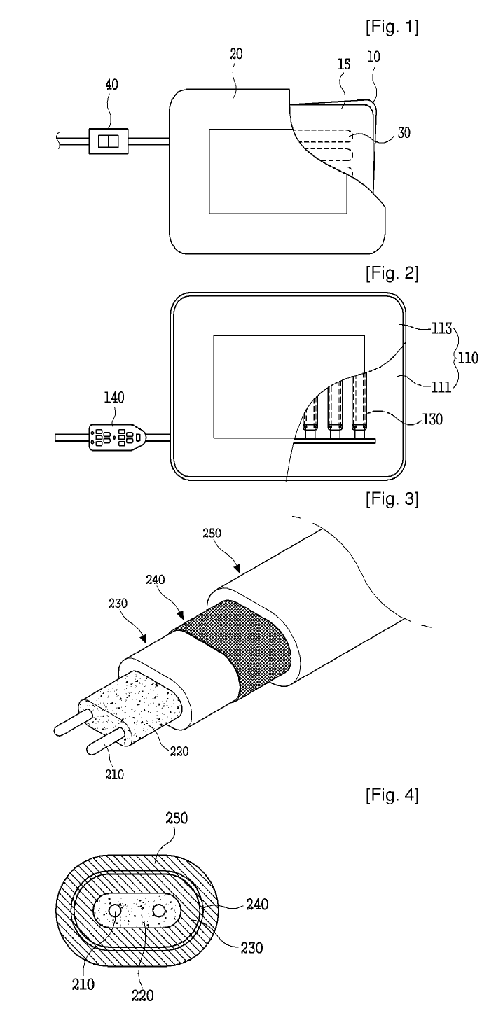

### MODE FOR THE INVENTION

Hereinafter, exemplary embodiments of the present invention will be described with reference to the accompanying drawings.

FIG. 2 is a partially cut-away perspective view illustrating a heating mat for preventing overheating according to the first embodiment of the present invention. FIG. 3 is a perspective view illustrating the interior structure of a self-regulating cable used as a heat generating means in the heating mat of the present invention. FIG. 4 is a cross-sectional view of the self-regulating cable illustrated in FIG. 3.

As illustrated in FIGS. 2 and 4, a heating mat for preventing overheating according to the present invention includes a heat generating means 130, a control unit 140, and an exterior material 110.

The heat generating means is a self-regulating cable having a function of maintaining a predetermined temperature by generating heat with applied electric power energy. In the heat generating means, a pair of wires 210 are arranged in the interior of an insulation film 230 by the medium of a semi-conductive polymer 220.

The semi-conductive polymer 220 is a heat generating body in which carbon is contained in a plastic resin and is a material having a self-regulating function due to its characteristics in which its resistance increases as its temperature increases. Further, if its temperature increases above a predetermined temperature, e.g. above 85 degrees Celsius, current cannot flow through the semi-conductive polymer like an insulation body.

If electric power is applied to a pair of wires 210 called bus wires, heat is generated by the resistance characteristics of the semi-conductive polymer 220 electrically connecting the bus wires and the peripheral temperature increases due to the heat generated in the semi-conductive polymer 220. If the temperature of the semi-conductive polymer 220 increases, the resistance of the semi-conductive polymer 220 increases and the amount of generated heat is reduced. Then, if the temperature of the semi-conductive polymer 220 reaches a predetermined temperature, current does not flow any more and generation of heat is stopped. Therefore, the temperature at the periphery of the semi-conductive polymer 220 decreases and the semi-conductive polymer 220 generates heat as a resistance body again.

Therefore, since the heat generating means 130 to which the semi-conductive polymer is applied has a function of preventing overheating by itself, it can basically solve the partial overheating problem generated in a conventional heating mat.

The above-mentioned function prevents the temperature of the semi-conductive polymer 220 from increasing above a predetermined temperature and the semi-conductive polymer 220 maintains a constant temperature and generates heat by the self-regulating function. Then, in the self-regulating cable, it is preferable that a ground wire 240 is formed outside an insulation film and the ground wire 240 and the insulation film 230 is surrounded by an external insulation film 250.

The cross-section of a general self-regulating cable is elliptic and the long axis of the ellipse is 10 to 20 mm thick. In the general self-regulating cable, it is difficult to arrange the thick self-regulating cable in zigzags to form a heating mat for preventing overheating. Further, in the conventional method, it is impossible to apply the general self-regulating cable to the heating mat and to use a heating mat using the self-regulating cable.

The present invention is adapted to solve the above-mentioned problem and bends a self-regulating cable applied to a heating mat.

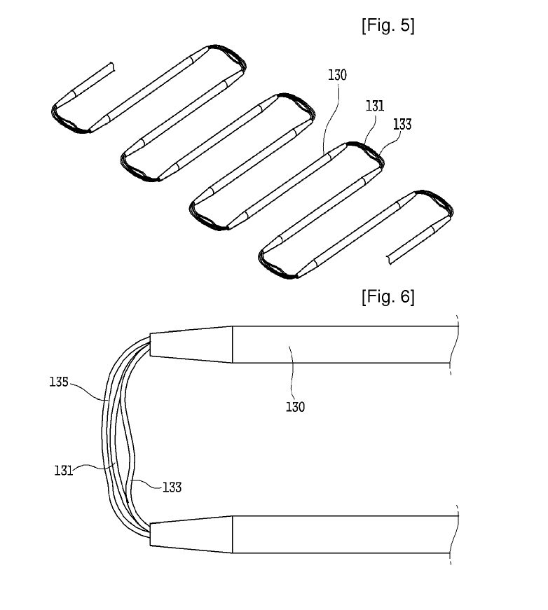

FIG. 5 is a perspective view illustrating the bent shape of the heat generating means used in the heating mat for preventing overheating illustrated in FIG. 2. FIG. 6 is a front view enlarging the bent portion of the heat generating means of FIG. 5.

As illustrated in FIGS. 5 and 6, in order to form bent portions in the self-regulating cable used as the heat generating means 130 used in the heating mat for preventing overheating according to the present invention, it is preferable that the thickness of the heat generating means 130 is partially different and portions of the heat generating means 130 which have a small thickness are bent.

Further, by applying the same method, it is preferable that in the heat generating means, a plurality of self-regulating cables are connected in series, and an engaging means is connected in series to the pair of wires of the self-regulating cable to connect a plurality of self-regulating cables. Then, the heat generating means is arranged in zigzags by bending the engaging means in the heat generating means.

Accordingly, as shown in FIGS. 5 and 6, it is preferable that the heat generating means 130 has a portion having a partially small thickness and the thin portion is easily bent. Further, it is preferable that the bus wires 131 and 133 which is a pair of wires are left together with a metal wire 135 to prevent leakage of current.

Since the thin portion can be easily bent as compared with the other portions, the self-regulating cable is arranged in zigzags to be applied to the heating mat for preventing overheating of the present invention.

For example, it is preferable that the self-regulating cable is exposed by partially removing the semi-conductive polymer and separating a pair of wires and at least one of the pair of exposed wires is surrounded by another insulation film. Then, it is preferable that the pair of exposed wires are bent and one of the exposed wires is connected to the ground. As illustrated in FIG. 6, it is preferable that when the ground wire is independently bent and used, the metal wire and the bus wires are surrounded by another insulation film to be insulated.

Further, it is preferable that in the self-regulating cable illustrated in FIGS. 3 and 4, the external insulation film 250, the insulation film 230, and the semi-conductive polymer 220 are removed at portions where the self-regulating cable is bent and the pair of wires 210 in the interior of the self-regulating cable are partially exposed to the outside. Then, it is preferable that at least one of the wires 210 is surrounded by another insulation film at the exposed portions to increase the insulation between the pair of wires 210.

It is preferable that the self-regulating cable is arranged in zigzags by removing the insulation film and the external insulation film and bending the portion having a small thickness. Then, it is preferable that the metal wire functioning as a ground wire 240 is left together with the bus wires which are the pair of wires 210 to prevent leakage of current.

The control unit 140 is electrically connected to the heat generating means 130 and has a function of supplying electric power to the wire of the self-regulating cable, i.e. the heat generating means 130 and of regulating the supplied electric power. Therefore, the control unit 140 is connected to an external power source to function as a power source supplying electric power to the self-regulating cable.

Then, it is preferable that the control unit 140 includes a current leakage interrupter detecting current leakage between the ground wire 240 and the pair of wires 210 and has a function interrupting generation of current leakage due to damage to the insulation film 230 during use.

It is preferable that the exterior material 110 surrounds the heat generating means 130 from outside and a surface of an upper portion 113 of the exterior material 110 is formed of a material making contact with the body of a user using the heating mat for preventing overheating according to the present invention and is formed of a fabric, e.g. a jacquard material so as to give a user a comfortable feeling.

Further, the exterior material 110 has a multi-layered structure using a fabric or leather and a lower portion 111 of the exterior material 110 provides a water-proof function and an insulation function to prevent generation of heat loss to the lower portion of the heat generating means.

It is preferable that an interior material (not illustrated) is inserted between the exterior material 110 and the heat generating means 130 and stone powder (not illustrated) including at least one material selected from a group consisting of germanium, tourmaline, loess, hardwood charcoal, crystal, elvan, and ceramic is provided between the interior material and the exterior material. The stone powder filled between the interior material and the exterior material 110 at an upper portion of the heat generating means 130 is converted into a embossed surface shape to provide a cushion to a user.

Since the heating mat for preventing overheating according to the present invention is arranged in zigzags in its interior by making the bent portion of the self-regulating cable used as the heat generating means 130 thin, a self-regulating cable which cannot be used conventionally is used as a heat generating body. Therefore, a heating mat for preventing temperature overheating of which is regulated to a constant temperature in the range of 10 to 85 degrees Celsius maintains the temperature of the heating mat for preventing overheating at a constant temperature regardless of the environment using the heating mat for preventing overheating. Further, the maximum temperature can be regulated by changing the condition of the self-regulating cable.

Further, carbon used as a main component material of the semi-conductive polymer 220 has a large emissivity of approximately 0.9. Since carbon is a material efficiently emitting far infrared rays having waves of 12 to 14 micrometers which are an excellent effect of curing the human body, it increases curing effects by far infrared rays.

Hereinafter, a method for bending a self-regulating cable to manufacture a heating mat for preventing overheating according to the present invention will be described.

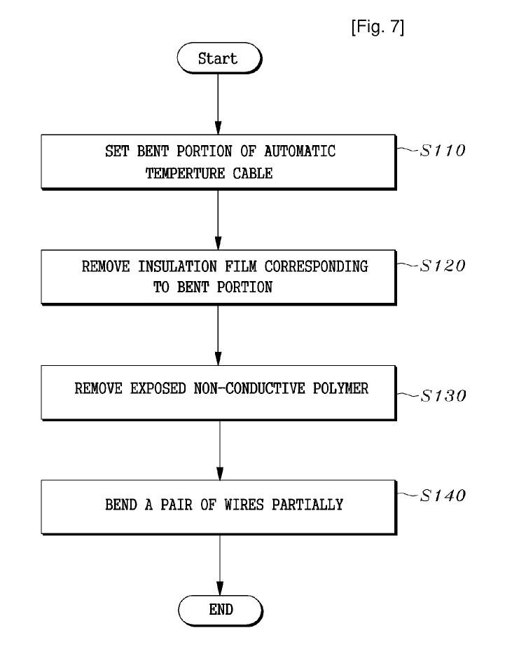

FIG. 7 is a flow chart for explaining a method for bending a self-regulating cable according to an embodiment of the present invention.

As illustrated in FIG. 7, a bending portion of a Self-regulating cable having a thick cross-section is set by the method for bending the self-regulating cable including a pair of wires arranged in the interior of an insulation film by the medium of a semi-conductive polymer filled in the interior of the insulation film (S100). It is preferable that a ground wire is formed outside the insulation film in the self-regulating cable and a ground wire and the insulation film are surrounded by an external insulation film to decide a portion to be bent according to the purpose of the self-regulating cable.

Thereafter, the external insulation film at a portion corresponding to the bent portion of the self-regulating cable is removed, and this step will be omitted in the case in which there does not exist the external insulation film.

Further, the semi-conductive polymer is exposed by removing the insulation film at a portion corresponding to the bent portion (S120), and a pair of wires are exposed by removing the exposed portion of the semi-conductive polymer electrically connecting and surrounding the pair of wires (S130).

As illustrated in FIG. 5, since portions of the self-regulating cable, from which the insulation film and the semi-conductive polymer, is sufficiently thin, it is possible to bend the self-regulating cable. Therefore, the self-regulating cable is bent by bending the exposed portion of the pair of wires in a wanted direction (S140). Then, it is preferable that at least one wire of the pair of exposed wires is surrounded using another insulation film to prevent the possibility of generating a short by the contact of the pair of wires from increasing.

The above-mentioned method for bending a Self-regulating cable according to the present invention can be used in a heating device or an industrial field by easily bending the self-regulating cable which has been difficult to bend and use by changing its direction.

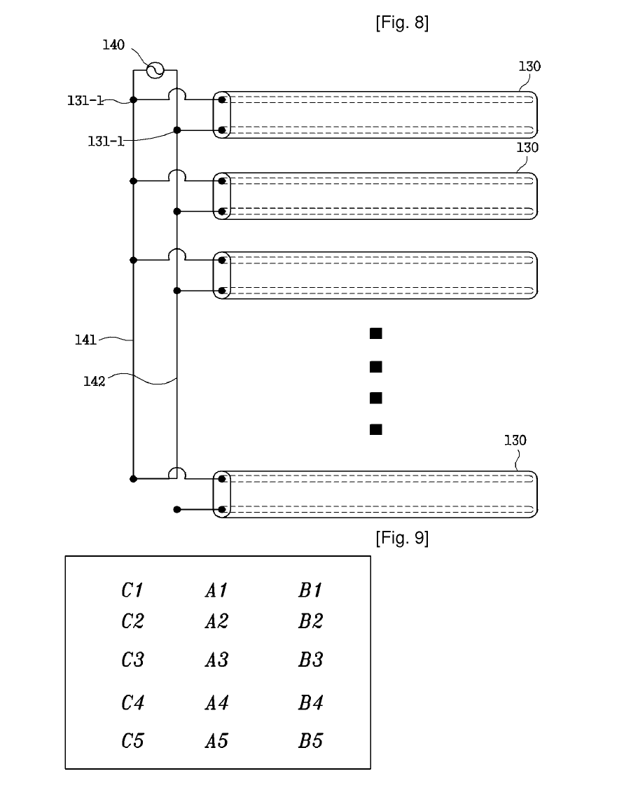

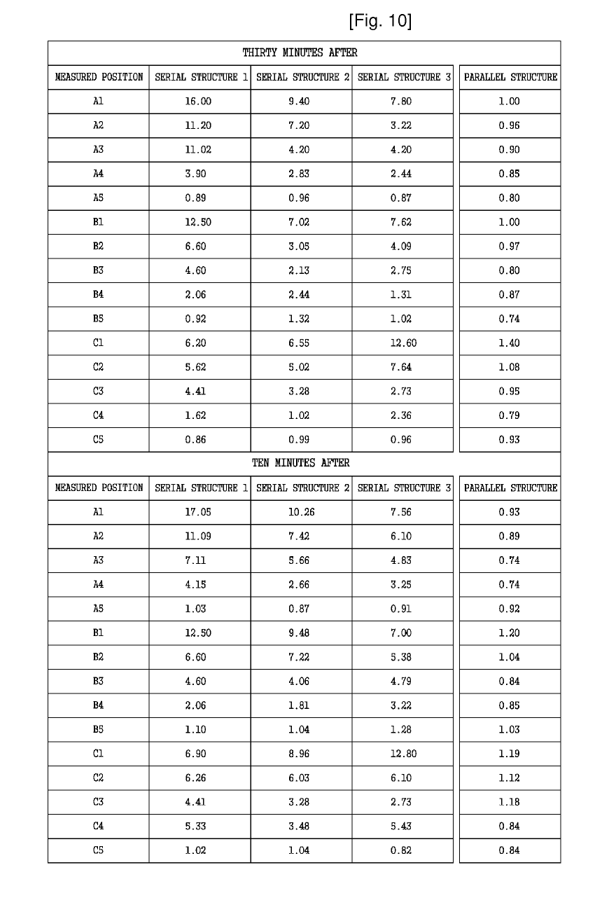

Meanwhile, FIG. 8 is a circuit connecting view illustrating a structure in which a heat generating means is connected in parallel to a heating mat according to another embodiment of the present invention. FIG. 9 is a plan view displaying positions where electromagnetic waves are measured in the heating mat illustrated in FIG. 8. FIG. 10 is a table displaying data showing a result obtained by measuring electromagnetic waves in mG (m Gauss) unit.

As illustrated in FIGS. 2, 4, 5, and 8, a heating mat for preventing overheating according to another embodiment of the present invention includes a plurality of heat generating means 130, a control unit 140, and an exterior material 110, and the plurality of heat generating means are connected in parallel.

As shown in FIG. 8, the plurality of heat generating means including a self-regulating cable having a temperature regulating function are branched out from electric wires 141 and 142 connected through the control unit 140 to a plurality of branch points 131-1 and are connected in parallel. The plurality of heat generating means are arranged in the interior of the exterior material. Then, the plurality of heat generating means functions as resistance bodies generating heat and are connected to the control unit in series.

The plurality of heat generating means divided in parallel to be connected are connected in parallel to reduce the current applied to the heat generating means, thereby reducing electromagnetic waves generated in the heat generating means.

Since if the parallel structure applied to the heating mat for preventing overheating according to the present invention is used, the bent portion of the self-regulating cable is eliminated, the heating mat for preventing overheating according to the present invention can be easily manufactured.

In the following, the electromagnetic waves generated in the heating mat for preventing overheating according to the present invention, which uses a Self-regulating cable, will be compared according to the structure in which the plurality of heat generating means are connected.

FIG. 9 is a plan view displaying positions where electromagnetic waves are measured in the heating mat illustrated in FIG. 8. FIG. 10 is a table displaying data showing a result obtained by measuring electromagnetic waves in mG (m Gauss) unit.

Then, the serial structure 1 is the case in which the self-regulating cable is bent by erecting the cable and the serial structures 2 and 3 are the cases in which the cable is bent by changing the thickness of the self-regulating cable. In the serial structure 2, a pair of wires in the interior of the self-regulating cable cross each other, and in the serial structure 3, they do not.

When comparing the results of FIG. 10, the self-regulating cable connected in series remarkably increases the electromagnetic waves according to its position. However, very small electromagnetic waves are measured in the self-regulating cable using a parallel structure.

Therefore, since the heating mat for preventing overheating according to the present invention has the characteristics in which if the peripheral temperature increases, the resistance of the heat generating means also increases, thereby automatically stopping generation of heat, it is prevented from being overheated above a predetermined temperature. Further, generation of electromagnetic waves are remarkably reduced to help the health of a user using the heating mat for preventing overheating.

### INDUSTRIAL APPLICABILITY

Although several exemplary embodiments of the present invention have been described for illustrative purposes, those skilled in the art will appreciate that various modifications, additions and substitutions are possible, without departing from the scope and spirit of the invention as disclosed in the accompanying claims.

1. A heating mat for preventing overheating, comprising:
a heat generating means for maintaining a predetermined temperature with applied electric power;
a control unit electrically connected to the heat generating means, for regulating the electric power supplied to the heat generating means; and
an exterior material surrounding the heat generating means.

2. A heating mat as claimed in claim 1, wherein the heat generating means is a self-regulating cable.

3. A heating mat as claimed in claim 2, wherein in the self-regulating cable, a pair of wires are arranged in the interior of an insulation film by the medium of a semi-conductive polymer.

4. A heating mat as claimed in claim 3, wherein the semi-conductive polymer comprises carbon.

5. A heating mat as claimed in claim 3, wherein in the self-regulating cable, a ground wire is formed on the exterior of the insulation film and the ground wire and the insulation film are surrounded by an external insulation film.

6. A heating mat as claimed in claim 2, wherein the heat generating means has a partially different thickness.

7. A heating mat as claimed in claim 6, wherein a portion of the heat generating means which has a small thickness is bent.

8. A heating mat as claimed in claim 6, wherein the heat generating means is exposed by removing a portion of the semi-conductive polymer and separating the pair of wires.

9. A heating mat as claimed in claim 8, wherein at least one of the pair of exposed wires is surrounded by another insulation film.

10. A heating mat as claimed in claim 8, wherein the pair of exposed wires are bent.

11. A heating mat as claimed in claim 8, wherein one of the pair of exposed wires is connected to the ground.

12. A heating mat as claimed in claim 5, wherein in the heat generating means, portions of the insulation film and the external insulation film, which are removed, are bent.

13. A heating mat as claimed in claim 12, wherein in the heat generating means, portions of the insulation film and the external insulation film, which are removed, are bent.

14. A heating mat as claimed in claim 12, wherein the heat generating means is exposed by removing a portion of the semi-conductive polymer and separating the pair of wires.

15. A heating mat as claimed in claim 14, wherein at least one of the pair of exposed wires is surrounded by another insulation film.

16. A heating mat as claimed in claim 14, wherein the ground wire separated from the pair of exposed wires is left.

17. A heating mat as claimed in claim 5, wherein the control unit comprises a current leakage interrupter detecting leaked current between the ground wire and the pair of wires.

18. A heating mat as claimed in claim 1, wherein in the heat generating means, a plurality of self-regulating cables are connected in series by an engaging means.

19. A heating mat as claimed in claim 18, wherein the engaging means is connected in series to the pair of wires of the self-regulating cable to connect a plurality of self-regulating cables.

20. A heating mat as claimed in claim 18, wherein the engaging means is bent in the heat generating means.

21. A heating mat as claimed in claim 1, further comprising:
an interior material surrounding the heat generating means in the interior of the exterior material.

22. A heating mat as claimed in claim 21, wherein stone powder is provided between the exterior material and the interior material.

23. A heating mat as claimed in claim 22, wherein the stone powder comprises at least one material selected from a group consisting of germanium, tourmaline, loess, hardwood charcoal, crystal, elvan, and ceramic.

24. A heating mat as claimed in claim 1, wherein a surface of the exterior material is a jacquard material.

25. A heating mat as claimed in claim 1, wherein the heat generating means is connected in parallel to a wire connected through the control unit and is arranged in the interior of the exterior material.

26. A method for bending a self-regulating cable comprising a pair of wires arranged in the interior of an insulation film by the medium of a semi-conductive polymer filled in the interior of the insulation film, the method comprising:
setting a bent portion of the self-regulating cable;
exposing the semi-conductive polymer by removing a portion of the insulation film, which corresponds to the bent portion;
exposing a pair of wires by removing the exposed semi-conductor polymer; and
bending portion of the pair of wires, which correspond to the exposed bent portion.

27. A method as claimed in claim 26, wherein the self-regulating cable comprises a ground wire formed outside the insulation film and an external insulation film surrounding the ground wire and the insulation film, the method further comprising:
removing a portion of the external film, which corresponds to the bent portion, after setting the bent portion of the self-regulating cable.

28. A method as claimed in claim 26, wherein at least one of the pair of wires is surrounded by another insulation film after exposing the pair of wires.
